(19) United States
(12) Patent Application Publication
(10) Pub. No.: US 2025/0073230 A1
(43) Pub. Date: Mar. 6, 2025

(54) USE OF PLINABULIN IN COMBINATION WITH IMMUNE CHECKPOINT INHIBITORS

(71) Applicant: BeyondSpring Pharmaceuticals, Inc., Florham Park, NJ (US)

(72) Inventors: Lan Huang, New York, NY (US); Gloria Tsi-Yie Lee, New York, NY (US)

(21) Appl. No.: 18/952,618

(22) Filed: Nov. 19, 2024

Related U.S. Application Data
(63) Continuation-in-part of application No. 15/550,350, filed on Aug. 10, 2017, now abandoned.
(60) Provisional application No. 62/115,468, filed on Feb. 12, 2015.
(60) Provisional application No. 62/255,259, filed on Nov. 13, 2015.

(51) Int. Cl. A61K 31/496; A61K 31/282; A61K 31/337; A61K 33/243; A61K 39/00; A61K 39/395; A61P 35/00

(52) U.S. Cl. CPC A61K 31/496; A61K 31/282; A61K 31/337; A61K 33/243; A61K 39/3955; A61P 35/00; A61K 2039/505

(57) ABSTRACT

Disclosed herein are compositions comprising Plinabulin and one or more immune checkpoint inhibitor for treating cancer. Some embodiments relate to methods of treating cancer by co-administering Plinabulin and one or more immune checkpoint inhibitor to a subject in need thereof.

### INCORPORATION BY REFERENCE TO ANY PRIORITY APPLICATIONS

This application is a continuation of U.S. application Ser. No. 15/550,350, filed Aug. 10, 2017, which is the U.S. National Phase of International Application No. PCT/US2016/017602, filed Feb. 11, 2016, which claims the benefit of U.S. Provisional Application No. 62/115,468, filed Feb. 12, 2015, and U.S. Provisional Application No. 62/255,259, filed Nov. 13, 2015. All of the foregoing applications are fully incorporated herein by reference in their entireties for all purposes.

### BACKGROUND

### Field

The present invention relates to the field of chemistry and medicine. More particularly, the present invention relates to Plinabulin, compositions containing Plinabulin, and its use in treatment.

### Description of the Related Art

Human cancers harbor numerous genetic and epigenetic alterations, generating neoantigens potentially recognizable by the immune system (Sjoblom et al, 2006). The adaptive immune system, comprised of T and B lymphocytes, has powerful anti-cancer potential, with a broad capacity and exquisite specificity to respond to diverse tumor antigens.

Recent cancer immunotherapy research has focused substantial effort on approaches that enhance anti-tumor immunity by adoptive-transfer of activated effector cells, immunization against relevant antigens, providing non-specific immune-stimulatory agents such as cytokines, or removing inhibitors to anti-cancer effector cells. Efforts to develop specific immune checkpoint inhibitors have begun to provide new immunotherapeutic approaches for treating cancer, including the development of an antibody, ipilimumab, that binds to and inhibits Cytotoxic T-Lymphocyte Antigen-4 (CTLA-4) for the treatment of patients with advanced melanoma (Hodi et al., 2010). While cancer remains as an incurable disease for the great majority of patients, there exists a particular need for developing effective therapeutic agents that can be used in cancer immunotherapy.

### SUMMARY OF THE INVENTION

Some embodiments relate to a pharmaceutical composition including Plinabulin and one or more immune checkpoint inhibitor.

Some embodiments relate to a method for treating cancer, the method including co-administering Plinabulin and one or more immune checkpoint inhibitor to a subject in need thereof.

### BRIEF DESCRIPTION OF THE DRAWINGS

FIG. 1A shows the expression of DC maturation markers CD40, CD80, CD86, and MHCII in dendritic cells treated with Plinabulin at various concentrations and with LPS control; FIG. 1B shows the viability of dendritic cells treated with Plinabulin and LPS.

FIG. 2A shows the expression of the CD40 marker in dendritic cells treated with Plinabulin, Paclitaxel, Etoposide, or control; FIG. 2B shows the expression of the CD80 marker in dendritic cells treated with Plinabulin, Paclitaxel, Etoposide, or control; FIG. 2C shows the expression of the CD86 marker in dendritic cells treated with Plinabulin, Paclitaxel, Etoposide, or control; FIG. 2D shows the expression of the MHCII marker in dendritic cells treated with Plinabulin, Paclitaxel, Etoposide, or control.

FIG. 3A shows the production of IL-10 in dendritic cells treated with Plinabulin, Paclitaxel. Etoposide, and control; FIG. 3B shows the production of IL-6 marker in dendritic cells treated with Plinabulin, Paclitaxel. Etoposide, and control; FIG. 3C shows the production of IL-12p40 in dendritic cells treated with Plinabulin, Paclitaxel. Etoposide, and control.

FIGS. 4A-4C show the plinabulin-induced enhancement of the anti-tumor effect of the PD-1 antibody plus CTLA-4 antibody in the MC-38 tumor model in immune competent mice. FIG. 4A shows the effect on tumor growth; FIG. 4B shows the effect on the mean tumor weight at necropsy; FIG. 4C shows the time for tumors to reach 10 fold of their starting volume.

FIGS. 5A-5C show the results of Fluorescence-activated cell sorting (FACS) analysis of the tumors at necropsy from the study described in Example 6. FIG. 5A shows the effect on Treg cells; FIG. 5B shows the ratio of CD8+ cells to Treg cells; FIG. 5C shows the effect on macrophages.

### DETAILED DESCRIPTION OF THE PREFERRED EMBODIMENT

Plinabulin, (3Z,6Z)-3-Benzylidene-6-{[5-(2-methyl-2-propanyl)-1H-imidazol-4-yl]methylene}-2,5-piperazinedione, is a synthetic analog of the natural compound phenylahistin. Plinabulin can be readily prepared according to methods and procedures detailed in U.S. Pat. Nos. 7,064,201 and 7,919,497, which are incorporated herein by reference in their entireties. In some embodiments, Plinabulin can efficiently promote antigen uptake and migration of dendritic cells to lymph nodes where tumor-specific antigens are presented by dendritic cells to prime immune effector cells. Exposure of dendritic cells to Plinabulin can induce maturation of dendritic cells and significantly increase their capacity to prime T cells. In some embodiments, Plinabulin can mediate tumor size reduction through immune modulation of the tumor microenvironment to promote anti-tumor immune enhancing effects. In some embodiments, substantial therapeutic synergies can be achieved when combining Plinabulin with immune checkpoint inhibitors.

Some embodiments relate to the use of Plinabulin in combination with one or more immune checkpoint inhibitors, such as inhibitors of CTLA4 (cytotoxic T lymphocyte antigen-4), PD-1 (programmed cell death protein 1), PD-L1 (programmed cell death ligand 1), PD-L2 (programmed cell death ligand 2), PD-L3 (programmed cell death ligand 3), PD-L4 (programmed cell death ligand 4), LAG-3 (lymphocyte activation gene-3), and TIM-3 (T cell immunoglobulin and mucin protein-3). In some embodiments, the immune checkpoint inhibitor is a binding ligand of PD-1. In some embodiments, the immune checkpoing inhibitor is a binding ligand of CTLA-4.

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. The term “PD-1” as used herein includes human PD-1 (hPD-1), variants, isoforms, and species homologs of hPD-1, and analogs having at least one common epitope with hPD-1.

Various cell surface glycoprotein ligands for PD-1 have been identified, including PD-L1, PD-L2, PD-L3, and PD-L4, that are expressed on antigen-presenting cells as well as many human cancers and have been shown to downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The term “PD-L2” as used herein includes human PD-L2 (hPD-L2), variants, isoforms, and species homologs of hPD-L2, and analogs having at least one common epitope with hPD-L2. The term “PD-L3” as used herein includes human PD-L3 (hPD-L3), variants, isoforms, and species homologs of hPD-L3, and analogs having at least one common epitope with hPD-L3. The term “PD-L4” as used herein includes human PD-L4 (hPD-L4), variants, isoforms, and species homologs of hPD-L4, and analogs having at least one common epitope with hPD-L4.

CTLA-4 (cytotoxic T-lymphocyte-associated protein 4) is a protein receptor that, functioning as an immune checkpoint, downregulates the immune system. CTLA4 is found on the surface of T cells, is also a member of the immunoglobulin (Ig) superfamily; CTLA-4 comprises a single extracellular Ig domain. CTLA-4 transcripts have been found in T cell populations having cytotoxic activity, suggesting that CTLA-4 might function in the cytolytic response.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety. The pharmaceutically acceptable excipient can be a monosaccharide or monosaccharide derivative.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

An “effective amount” or a “therapeutically effective amount” as used herein refers to an amount of a therapeutic agent that is effective to relieve, to some extent, or to reduce the likelihood of onset of, one or more of the symptoms of a disease or condition, and can include curing a disease or condition.

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, the term “chemotherapeutic agent” refers to an agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents include but are not limited to, for example, fluoropyrimidines; pyrimidine nucleosides; purine nucleosides; anti-folates, platinum-based agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

### Administration and Pharmaceutical Compositions

Some embodiments relate to a pharmaceutical composition, comprising Plinabulin and one or more immune checkpoint inhibitor.

In some embodiments, the immune checkpoint inhibitor is an inhibitor of PD-1, PD-L1, PD-L2, PD-L3, PD-L4, CTLA-4, LAG3, B7-H3, B7-H4, KIR or TIM3. In some embodiments, the immune checkpoint inhibitor is a PD-1 inhibitor. In some embodiments, the immune checkpoint inhibitor is a binding ligand of PD-L1. In some embodiments, the immune checkpoint inhibitor is a PD-L1 inhibitor. In some embodiments, the immune checkpoint inhibitor is a PD-L2 inhibitor or a combined PD-L1/PD-L2 inhibitor. In some embodiments, the immune checkpoint inhibitor is a CTLA-4 inhibitor.

In some embodiments, the composition described herein includes a first immune checkpoint inhibitor and a second immune checkpoint inhibitor, wherein the first immune checkpoint inhibitor is different from the second immune checkpoint inhibitor. In some embodiments, the first and the second immune checkpoint inhibitor is independently an inhibitor of PD-1, PD-L1, PD-L2, PD-L3, PD-L4, CTLA-4, LAG3, B7-H3, B7-H4, KIR or TIM3. In some embodiments, the first immune checkpoint inhibitor is a PD-1 inhibitor, and the second immune checkpoint inhibitor is a CTLA-4 inhibitor. In some embodiments, the first immune checkpoint inhibitor is a PD-L1 inhibitor, and the second immune checkpoint inhibitor is a CTLA-4 inhibitor. In some embodiments, the first immune checkpoint inhibitor is a PD-L2 inhibitor, and the second immune checkpoint inhibitor is a CTLA-4 inhibitor.

In some embodiments, the immune checkpoint inhibitor can be a small peptide agent that can inhibit T cell regulation function. In some embodiments, the immune checkpoint inhibitor can be a small molecule (e.g. less than 500 Daltons) that can inhibit T cell regulation function. In some embodiments, the immune checkpoint inhibitor can be a molecule providing co-stimulation of T-cell activation. In some embodiments, the immune checkpoint inhibitor can be a molecule providing co-stimulation of natural killer cell activation. In some embodiments, the immune checkpoint inhibitor can be an antibody. In some embodiments, the immune checkpoint inhibitor is a PD-1 antibody. In some embodiments, the immune checkpoint inhibitor is a PD-L1 antibody. In some embodiments, the immune checkpoint inhibitor is a PD-L2 antibody. In some embodiments, the immune checkpoint inhibitor is a PD-L3 antibody. In some embodiments, the immune checkpoint inhibitor is a PD-L4 antibody. In some embodiments, the immune checkpoint inhibitor is a CTLA-4 antibody. In some embodiments, the immune checkpoint inhibitor is an antibody of CTLA-4, LAG3, B7-H3, B7-H4, KIR, or TIM3.

The antibody can be selected from α-CD3-APC, α-CD3-APC-H7, α-CD4-ECD, α-CD4-PB, α-CD8-PE-Cy7, α-CD-8-PerCP-Cy5.5, α-CD11c-APC, α-CD11b-PE-Cy7, α-CD11b-AF700, α-CD14-FITC, α-CD16-PB, α-CD19-AF780, α-CD19-AF700, α-CD20-PO, α-CD25-PE-Cy7, α-CD40-APC, α-CD45-Biotin, Streptavidin-BV605, α-CD62L-ECD, α-CD69-APC-Cy7, α-CD80-FITC, α-CD83-Biotin, Streptavidin-PE-Cy7, α-CD86-PE-Cy7, α-CD86-PE, α-CD123-PE, α-CD154-PE, α-CD161-PE, α-CTLA4-PE-Cy7, α-FoxP3-AF488 (clone 259D), IgG1-isotype-AF488, α-ICOS (CD278)-PE, α-HLA-A2-PE, α-HLA-DR-PB, α-HLA-DR-PerCPCy5.5, α-PD1-APC, VISTA, co-stimulatory molecule OX40, and CD137.

A variety of antibodies (Abs) can be used in the composition described herein, including antibodies having high-affinity binding to PD-1 PD-L1, PD-L2, PD-L3, or PD-L4. Human mAbs (HuMAbs) that bind specifically to PD-1 (e.g., bind to human PD-1 and may cross-react with PD-1 from other species, such as cynomolgus monkey) with high affinity have been disclosed in U.S. Pat. No. 8,008,449, which is incorporated herein by reference in its entirety. HuMAbs that bind specifically to PD-L1 with high affinity have been disclosed in U.S. Pat. No. 7,943,743, which is incorporated herein by reference in its entirety. Other anti-PD-1 mAbs have been described in, for example, U.S. Pat. Nos. 6,808,710, 7,488,802 and 8,168,757, and PCT Publication No. WO 2012/145493, all of which are incorporated herein by reference in their entireties. Anti-PD-L1 mAbs have been described in, for example, U.S. Pat. Nos. 7,635,757 and 8,217,149, U.S. Publication No. 2009/0317368, and PCT Publication Nos. WO 2011/066389 and WO 2012/14549, all of which are incorporated herein by reference in their entireties.

In some embodiments, the anti-PD-1 HuMAbs can be selected from 17D8, 2D3, 4H1, 5C4 (also referred to herein as nivolumab), 4A1 1, 7D3 and 5F4, all of which are described in U.S. Pat. No. 8,008,449. In some embodiments, the anti-PD-1 HuMAbs can be selected from 3G10, 12A4 (also referred to herein as BMS-936559), 10A5, 5F8, 10H10, 1B12, 7H1, 1 1E6, 12B7, and 13G4, all of which are described in U.S. Pat. No. 7,943,743.

In some embodiments, the composition can further include one or more pharmaceutically acceptable diluents. In some embodiments, the pharmaceutically acceptable diluent can include Kolliphor HS15® (Polyoxyl (15)-hydroxystearate). In some embodiments, the pharmaceutically acceptable diluent can include propylene glycol. In some embodiments, the pharmaceutically acceptable diluents can include kolliphor and propylene glycol. In some embodiments, the pharmaceutically acceptable diluents can include kolliphor and propylene glycol, wherein the kolliphor is about 40% by weight and propylene glycol is about 60% by weight based on the total weight of the diluents. In some embodiments, the composition can further include one or more other pharmaceutically acceptable excipients.

Standard pharmaceutical formulation techniques can be used to make the pharmaceutical compositions described herein, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of Plinabulin or pharmaceutically acceptable salts thereof; (b) an immune checkpoint inhibitor and (c) a pharmaceutically acceptable carrier, diluent, excipient or combination thereof.

Other embodiments include co-administering Plinabulin and one or more immune checkpoint inhibitor in separate compositions. Thus, some embodiments include a first pharmaceutical compositions comprising: (a) a safe and therapeutically effective amount of Plinabulin or pharmaceutically acceptable salts thereof and (b) a pharmaceutically acceptable carrier, diluent, excipient or combination thereof; and a second pharmaceutical composition comprising: (a) one or more immune checkpoint inhibitor and (b) a pharmaceutically acceptable carrier, diluent, excipient or combination thereof.

Administration of the pharmaceutical compositions described herein can be via any of the accepted modes of administration for agents that serve similar utilities including, but not limited to, orally, sublingually, buccally, subcutaneously, intravenously, intranasally, topically, transdermally, intradermally, intraperitoneally, intramuscularly, intrapulmonarilly, vaginally, rectally, or intraocularly. Oral and parenteral administrations are customary in treating the indications that are the subject of the preferred embodiments.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The compositions described herein are preferably provided in unit dosage form. As used herein, a “unit dosage form” is a composition containing an amount of a compound or composition that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, although a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, sublingual, buccal, nasal, rectal, topical (including transdermal and intradermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound or composition. The amount of carrier employed in conjunction with the compound or composition is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules (e.g. solid gel capsules and liquid gel capsules), granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject composition is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the composition disclosed herein are employed. Topical formulations may generally be comprised of a pharmaceutical carrier, co-solvent, emulsifier, penetration enhancer, preservative system, and emollient.

For intravenous administration, the compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan. In some embodiments, a daily dose of Plinabulin may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day.

In some embodiments, the compositions described herein can be used in combination with other therapeutic agents. In some embodiments, the compositions described herein can be administered or used in combination with treatments such as chemotherapy, radiation, and biologic therapies.

### Method of Treatment

Some embodiments relate to a method for treating cancer using the pharmaceutical composition described herein to a subject in need thereof. Some embodiments relate to a method for treating cancer, comprising co-administering Plinabulin and one or more immune checkpoint inhibitor to a subject in need thereof. In some embodiments, the subject can be an animal, e.g., a mammal, a human. In some embodiments, the subject is a human.

Some embodiments relate to methods of providing co-stimulation of T-cell activation against cancer by co-administering plinabulin and one or more immune checkpoint inhibitor. Some embodiments relate to methods of providing co-stimulation of natural killer cells against cancer by co-administering plinabulin and one or more immune checkpoint inhibitor.

In some embodiments, the cancer comprises cancer cells expressing a binding ligand of PD-1. In some embodiments, the binding ligand of PD-1 is PD-L1. In some embodiments, the binding ligand of PD-1 is PD-L2.

In some embodiments, the method of treating cancer described herein further includes identifying cancer cells expressing a binding ligand of PD-1. In some embodiments, the method of treating cancer described herein further includes identifying cancer cells expressing PD-L1. In some embodiments, the method of treating cancer described herein further includes identifying cancer cells expressing PD-L2. In some embodiments, the method of treating cancer described herein further includes identifying cancer cells expressing PD-L3 or PD-L4.

In some embodiments, identifying cancer cells expressing a binding ligand of PD-1 includes using an assay to detect the presence of the binding ligand. Examples of applicable assay include but are not limited to PD-L1 IHC 22C3 pharmDx kit and PD-L1 IHC 28-8 pharmDx available from Dako.

In some embodiments, the cancer comprises cancer cells expressing a binding ligand of CTLA-4. In some embodiments, the binding ligand of CTLA-4 is B7.1 or B7.2.

In some embodiments, the method of treating cancer described herein further includes identifying cancer cells expressing a binding ligand of CTLA-4. In some embodiments, the method of treating cancer described herein further includes identifying cancer cells expressing B7.1 or B7.2.

In some embodiments, the immune checkpoint inhibitor is nivolumab, pembrolizumab, pidilizumab, ipilimumab, dacarbazine, BMS 936559, atezolizumab, durvalimumab, or any combinations thereof.

In some embodiments, cancer is head and neck cancer, lung cancer, stomach cancer, colon cancer, pancreatic cancer, prostate cancer, breast cancer, kidney cancer, bladder cancer, ovary cancer, cervical cancer, melanoma, glioblastoma, myeloma, lymphoma, or leukemia. In some embodiments, the cancer is renal cell carcinoma, malignant melanoma, non-small cell lung cancer (NSCLC), ovarian cancer, Hodgkin's lymphoma or squamous cell carcinoma. In some embodiments, the cancer is selected from breast cancer, colon cancer, rectal cancer, lung cancer, prostate cancer, melanoma, leukemia, ovarian cancer, gastric cancer, renal cell carcinoma, liver cancer, pancreatic cancer, lymphomas and myeloma. In some embodiments, the cancer is a solid tumor or hematological cancer.

In some embodiments, the cancer does not have any cells expressing PD-1, PD-L1, or PD-L2 at detectable levels.

In some embodiments, the cancer is selected from breast cancer, colon cancer, rectal cancer, lung cancer, prostate cancer, melanoma, leukemia, ovarian cancer, gastric cancer, renal cell carcinoma, liver cancer, pancreatic cancer, lymphomas and myeloma. In some embodiments, the cancer is a solid tumor or hematological cancer.

Some embodiments relate to a method of inducing dendritic cell maturation in a cancer patient, comprising administering to a composition comprising Plinabulin to a cancer patient.

Some embodiments relate to a method of disrupting cancer associated tumor vasculature in a subject comprising co-administering to the subject a compound of plinabulin and one or more immune checkpoint inhibitor.

Various cancers are associated the formation of tumor vasculature. In some embodiments, the cancer is selected from the group consisting of a melanoma, a pancreatic cancer, a colorectal adenocarcinoma, a brain tumor, acute lymphoblastic leukemia, chronic lymphocytic leukemia, hormone refractory metastatic prostate cancer, metastatic breast cancer, non-small cell lung cancer, renal cell carcinoma, head and neck cancer, prostate cancer, colon cancer, anaplastic thyroid cancer.

Some embodiments include co-administering a composition, and/or pharmaceutical composition described herein, with an additional medicament. For example, as described above, some embodiments include co-administering Plinabulin with one or more immune checkpoint inhibitor. By “co-administration,” it is meant that the two or more agents are administered in such a manner that administration of one or more agent has an effect on the efficacy and/or safety of the one or more other agent, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally or intravenously. In another embodiment, the agents are administered through different routes, such as one being administered orally and another being administered i.v. In some embodiments, the time period between administration of one or more agent and administration of the co-administered one or more agent can be about 1 hour, 2 hours, 3 hours, 5 hours, 8 hours, 10 hours, 12 hours, 15 hours, 18 hours, 20 hours, 24 hours, 36 hours, 48 hours, 3 days, 4 days, 5 days, 6 days, 7 days, 10 days, 14 days, 21 days, 28 days, or 30 days.

In some embodiments, the treatment cycle can include co-administering Plinabulin and one or more immune checkpoint inhibitors in combination with administering Plinabulin alone or administering one or more checkpoint inhibitor alone. In some embodiments, plinabulin and one or more immune checkpoint inhibitor are co-administered on day 1, followed by administration of plinabulin alone after 1 day, 2 days, 3 days, 4 days, 5 days, 6 days, 7 days, 2 weeks, or 3 weeks, and then followed by co-administration of plinabulin and one or more immune checkpoint inhibitor after 1 day, 2 days, 3 days, 4 days, 5 days, 6 days, 7 days, 2 weeks, or 3 weeks. In some embodiments, plinabulin and one or more immune checkpoint inhibitor are administered simultaneously on day 1, followed by administration of plinabulin or one or more immune checkpoint inhibitor alone on a day selected between day 2 and day 31, and then followed by co-administration of plinabulin and one or more immune checkpoint inhibitor on a day selected between day 3 and day 31. In some embodiments, plinabulin and one or more immune checkpoint inhibitor are co-administered on day 1, followed by administration of plinabulin alone on day 8, and then followed by co-administration of plinabulin and one or more immune checkpoint inhibitor on day 15. In some embodiments, the treatment cycle can be repeated two or more times.

Examples of additional medicaments include other chemotherapeutic agents.

In some embodiments, the chemotherapeutic agent can be selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Cabazitaxel, Cabozantinib-S-Malate, CAF, Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cobimetinib, Cometriq (Cabozantinib-S-Malate), COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idelalisib, Ifex (Ifosfamide), Ifosfamide, IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Netupitant and Palonosetron Hydrochloride, Neupogen (Filgrastim), Nexavar (Sorafenib Tosylate), Nilotinib, Ninlaro (Ixazomib Citrate), Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, Pegaspargase, Peginterferon Alfa-2b, PEG-Intron (Peginterferon Alfa-2b), Pembrolizumab, Pemetrexed Disodium Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituximab, Rolapitant Hydrochloride, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thioguanine, Thiotepa, Tolak (Fluorouracil—Topical), Toposar (Etoposide), Topotecan Hydrochloride, Toremifene, Torisel (Temsirolimus), Tositumomab and Iodine I 131, Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### EXAMPLES

### Example 1. Plinabulin Effect on Dendritic Cell Maturation

Cell lines: The immature mouse DC cell line SP37A3 (provided by Merck KGaA) was cultured in Iscove's Modified Dulbecco's Medium (IMDM; Sigma) supplemented with 10% heat-inactivated and endotoxin-tested FBS (PAA), sodium pyruvate (Gibco), penicillin/streptomycin L-glutamine mix (Gibco), Eagle's Minimum Essential Medium (MEM) nonessential amino acids (Sigma), Ciproxin (Bayer), and 0.05 mmol/L 2-mercaptoethanol (Gibco). IMDM complete medium was supplemented with 20 ng/mL recombinant mouse GM-CSF and 20 ng/mL recombinant mouse M-CSF (both Peprotech). The murine tumor cell lines EG7 and 3LL-OVA were obtained from ATCC or provided by Douglas T. Fearon (Cancer Research UK Cambridge Institute, Li Ka Shing Centre, University of Cambridge, Cambridge, UK), respectively. All cell lines were tested and validated to be Mycoplasma-free. Expression of OVA in EG7 and 3LL-OVA, and of Thy1.1 in RMAThy1.1, respectively, was confirmed; no genomic authentication was performed.

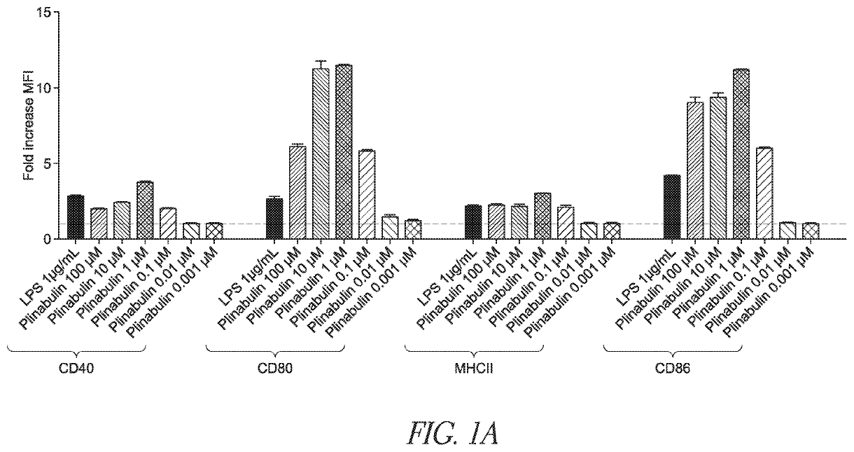

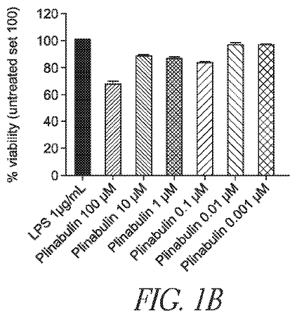

SP37A3 DCs (murine DC line, Merck) were plated (8×10⁴ cells/well, 96-well flat bottom, tissue-culture treated) in 180 uL IMDM complete medium [IMDM medium (Sigma) supplemented with 10% heat-inactivated and endotoxin-tested FBS (PAA), sodium pyruvate (Gibco), penicillin/streptomycin L-glutamine mix (Gibco), MEM nonessential amino acids (Sigma) and 0.05 mM 2-mercaptoethanol (Gibco)]. IMDM complete medium was supplemented with 20 ng/mL recombinant mouse GM-CSF. DCs were allowed to adhere for two hours before Plinabulin, medium, or LPS as controls were added 10× concentrated in 20 uL. DCs were incubated with Plinabulin in various concentrations (0.001 μM, 0.01 μM, 0.1 μM, 1 μM, 10 μM), medium, and LPS respectively for 20 h. Supernatants of these cultures were collected and used for detection of cytokine production by ELISA (kits available from BD) and the cells were stained with the LD-IR viability dye (Invitrogen) as well as with fluorochrom-labeled monoclonal antibodies against CD80, CD86, CD40 and MHCII for flow cytometric analysis. Cells were analyzed using a BD Fortessa Cytometer equipped with DIVA software. Mean fluorescence intensity (MFI) of the DC maturation markers CD40, CD80, CD86 and MHCII in live cells was normalized to the MFI of those markers detected in untreated (medium) DCs. As shown in FIG. 1A, Plinabulin significantly increased expression of all four DC maturation markers: CD40, CD80, CD86 and MHCII. DC viability did not change significantly at any of the drug concentrations tested, as determined using SytoxGreen staining, as shown in FIG. 1B.

### Example 2. Plinabulin in Comparison with Paclitaxel and Etoposide on Dendritic Cell Maturation

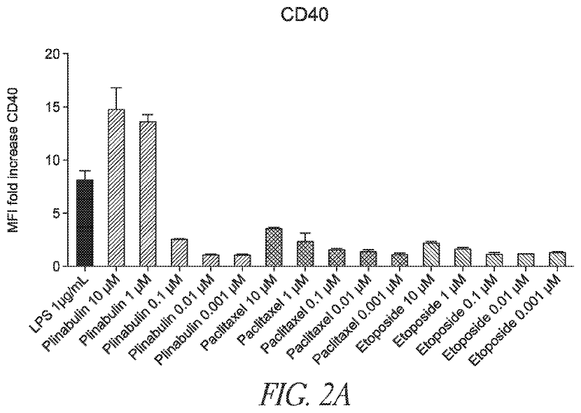

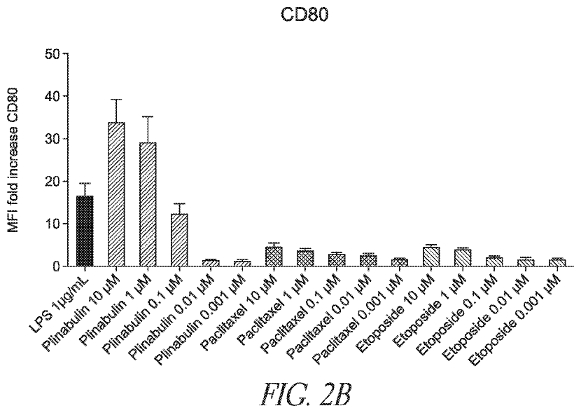

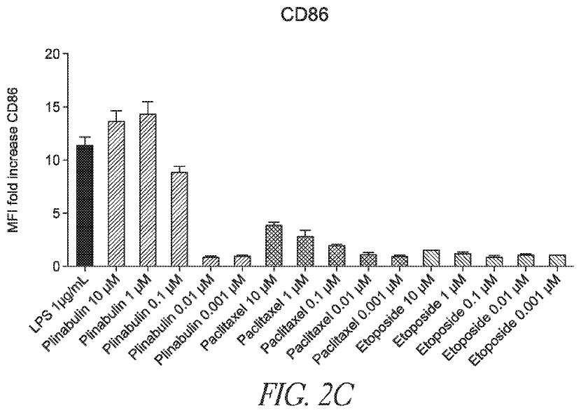

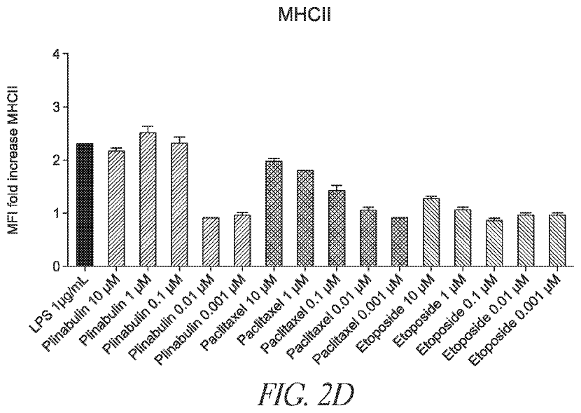

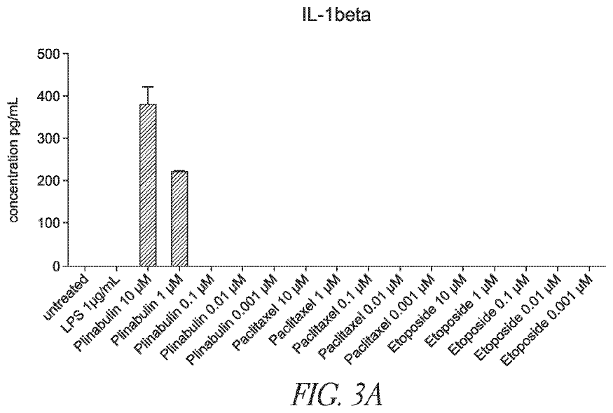

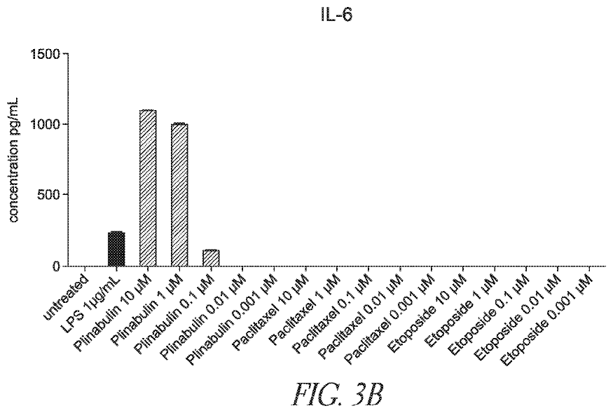

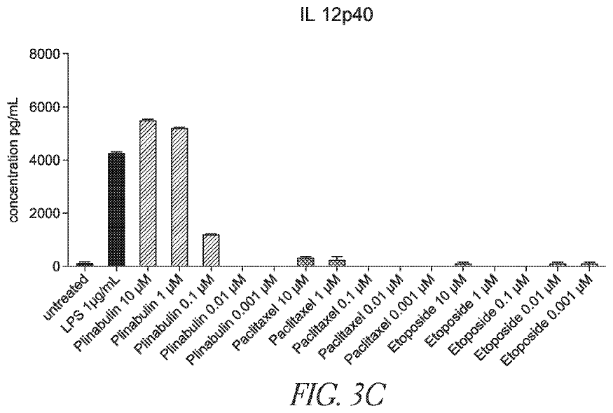

Two other cancer drugs, Paclitaxel and Etoposide, were also tested to compare their effects on DC maturation with Plinabulin. SP37A3 DCs (murine DC line, Merck) were plated (8×10⁴ cells/well, 96-well flat bottom, tissue-culture treated) in 180 uL IMDM complete medium [IMDM medium (Sigma) supplemented with 10% heat-inactivated and endotoxin-tested FBS (PAA), sodium pyruvate (Gibco), penicillin/streptomycin L-glutamine mix (Gibco), MEM nonessential amino acids (Sigma) and 0.05 mM 2-mercaptoethanol (Gibco)]. IMDM complete medium was supplemented with 20 ng/mL recombinant mouse GM-CSF. DCs were allowed to adhere for two hours before Plinabulin, Paclitaxel, Etoposide, medium, or LPS (positive control) were added 10× concentrated in 20 uL. DCs were incubated with Plinabulin (0.001 μM, 0.01 μM, 0.1 μM, 1 μM, 10 μM), Paclitaxel (0.001 μM, 0.01 μM, 0.1 μM, 1 μM, 10 μM), Etoposide (0.001 μM, 0.01 μM, 0.1 μM, 1 μM, 10 μM), medium, and LPS (positive control) respectively for 20 h. Supernatants of these cultures were collected and used for detection of cytokine production by ELISA (kits available from BD) and the cells were stained with the LD-IR viability dye (Invitrogen) as well as with fluorochrom-labeled monoclonal antibodies against CD80, CD86, CD40 and MHCII for flow cytometric analysis. Cells were analyzed using a BD Fortessa Cytometer equipped with DIVA software. Mean fluorescence intensity (MFI) of the DC maturation markers CD40 (FIG. 2A), CD80 (FIG. 2B), CD86 (FIG. 2C) and MHCII (FIG. 2D) in live cells was normalized to the MFI of those markers detected in untreated (medium) DCs. The production of the pro-inflammatory cytokines IL-1β(FIG. 3A), IL-6 (FIG. 3B), and IL-12p40 (FIG. 3C) were also determined by ELISA. Supernatants from the DC cultures were analyzed for these proinflammatory cytokines that have been demonstrated to play critical roles in regulating T-cell function and antitumor immune responses.

It was noted that Plinabulin was the most potent inducer of DC maturation among all three drugs. Plinabulin showed much greater expression of all four DC maturation markers, CD 40, CD 80, MHCII, and CD 86 than Paclitaxel and Etoposide. Plinabulin also showed significantly increased expression of all four markers when compared with the positive control LPS. Plinabulin triggered increased production of IL1b, IL6, and IL12, compared to in contrast to Paclitaxel, Etoposide, and LPS. Therefore, Plinabulin increased up-regulation of maturation markers and production of pro-inflammatory cytokines, resulting in an enhanced T cell stimulatory capacity.

### Example 3. Synergy of Plinabulin and Immune Checkpoint Inhibitors (PD-1 Antibody)

The combined treatment with Plinabulin and a PD-1 checkpoint inhibitor is tested in comparison with the treatment with Plinabulin alone and the treatment with PD-1 antibody alone. The tests are performed using seven to ten-week old mice that are injected subcutaneously with MC-38 tumor cells. Five testing groups are prepared, and each group includes 9 mice.

Group 1 is administered with saline; Group 2 is administered with the Plinabulin diluent (in the absence of Plinabulin); Group 3 is administered with Plinabulin dissolved in diluent at a concentration of 7.5 mg/kg; Group 4 is administered with PD-1 antibody; and Group 5 is administered with a Plinabulin/PD-1 antibody combined treatment. For the Plinabulin/PD-1 antibody combined treatment (Group 5), the mice are administered twice per week (Day 1 and Day 4 of each week) with Plinabulin (7.5 mg/kg) that is dissolved in diluent, followed by administering PD-1 antibody one hour after each Plinabulin administration. For the Plinabulin only treatment (Group 3) or the antibody only treatment (Group 4), mice are administered Plinabulin (7.5 mg/kg dissolved in diluent) or antibody alone twice per week (Day 1 and Day 4 of each week). For Groups 1 and 2, the mice are administered with saline or the Plinabulin diluent alone twice per week.

Each treatment starts at tumor size of around 125 mm³ and continues until tumor size of 1500 mm³ is reached. If the mean tumor size in any group has not reached 1500 mm³ by Experimental Day 45, treatment will be stopped and tumor size continued to be assessed. To determine the efficacy of each treatment, the following data are collected: mortality rate prior to tumor size reaching 1500 mm³; the body weight of the mice assessed twice weekly both prior to treatments; the rate of tumor growth as determined by the tumor size measurement (twice every week); the tumor growth index; overall survival rate; and the time required to double tumor size. The test results of the combined treatment with Plinabulin and PD-1 antibody show that Plinabulin acts in synergy with PD-1 antibody in inhibiting tumor growth.

### Example 4. In Vivo Stimulation of OVA Specific OT-I and OT-II T Cells

SP37A3 cells or day 7 BMDCs are pulsed for 1 hour with OVA full-length protein (0.1 mg/mL) before activation with Plinabulin or with OVA257-264 peptide (T4)/OVA323-339 peptide (500 ng/mL; after activation) and added at the indicated ratios to CD8⁺/CD4⁺T cells purified from OT-I/OT-II transgenic mice (2×10⁵ total cells/well, 96-well round bottomed plate). CD4⁺ T cells are loaded with the proliferation dye eFluor670 before co-culture. Proliferation is assessed after 3 days using flow cytometry.

### Example 5. In Vivo Stimulation of Antigen Specific CD4 and CD8 T Cells

Langerhans cells (LC) and spleen cells from naive OT-I and OT-II transgenic mice (Ly5.2) are labeled with eFluor670 and adoptively transferred into C57BL/6-Ly5.1 mice. After 24 hours, mice are immunized via tail-base injection with OVA257-264 peptide (T4: SIINFEKL; low-affinity variant of SIINFEKL) or OVA323-339 peptide together with Plinabulin or LPS. Proliferation of OT-I CD8⁺ and OT-II CD4⁺ T cells is assessed 4 days after adoptive transfer by flow cytometry.

### Example 6. Analysis of DC Homing to Tumor Draining LNs

For detection of DC homing upon injection of Plinabulin, mice bearing subcutaneous EG7 tumors are injected intratumorally with FITC-conjugated dextran (100 mg/mouse; Sigma) together with Plinabulin or PBS/carrier (mock control). Single-cell suspensions from tumor draining and nondraining LNs are prepared 48 hours after injection of Plinabulin and analyzed by flow cytometry.

### Example 7. Synergy of Plinabulin and Immune Checkpoint Inhibitors (PD-1 Antibody and CTLA-4 Antibody)

The combined treatment with Plinabulin and a PD-1 checkpoint inhibitor in combination with a CTLA-4 checkpoint inhibitor was tested in comparison with the treatment with Plinabulin alone, the treatment with PD-1 antibody alone, or the treatment with PD-1 antibody in combination with CTLA-4 antibody. The tests were performed using seven to ten-week old mice that were injected subcutaneously with MC-38 tumor cells. Six testing groups were prepared, and each group included 10 mice.

Group 1 was administered with IgG2a and plinabulin vehicle; Group 2 was administered with Plinabulin dissolved in diluent at a concentration of 7.5 mg/kg; Group 3 was administered with PD-1 antibody; Group 4 was administered with a Plinabulin/PD-1 antibody combined treatment; Group 5 was administered combined PD-1/CTLA-4 antibodies; and Group 6 was administered combined PD-1 antibody/CTLA-4 antibody/Plinabulin treatment. For the Plinabulin/PD-1 antibody combined treatment (Group 4) and the Plinabulin/PD-1/CTLA-4 antibody treatment (Group 6), the mice were administered twice per week (Day 1 and Day 4 of each week) with Plinabulin (7.5 mg/kg) that was dissolved in diluent, followed by administering antibody (ies) one hour after each Plinabulin administration. For the Plinabulin only treatment (Group 2) or the antibody (ies) only treatment (Groups 3 and 5), mice were administered Plinabulin (7.5 mg/kg dissolved in diluent) or antibody (ies) alone twice per week (Day 1 and Day 4 of each week).

Each treatment started at tumor size of around 125 mm³ and continued until tumor size of 3000 mm³ was reached. When the mean tumor size for Group 1 reached 3000 mm³, the experiment ended. To determine the efficacy of each treatment, the following data were collected: mortality rate prior to tumor size reaching 3000 mm³; the body weight of the mice assessed twice weekly both prior to treatments; the rate of tumor growth as determined by the tumor size measurement (twice every week); the tumor growth index; overall survival rate; the tumor weight at necropsy; and the time required to increase tumor size 10 fold. At necropsy the tissues were weighed and subjected to FACS analysis.

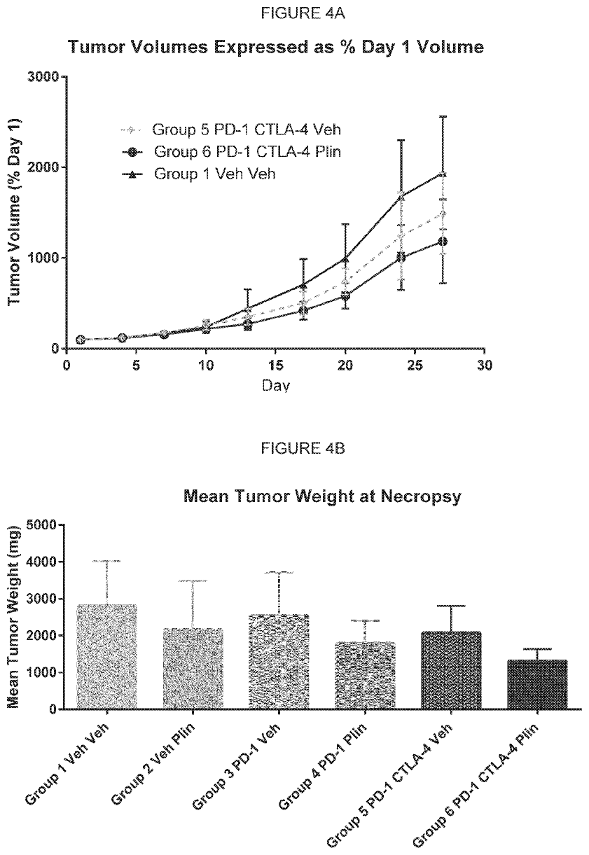

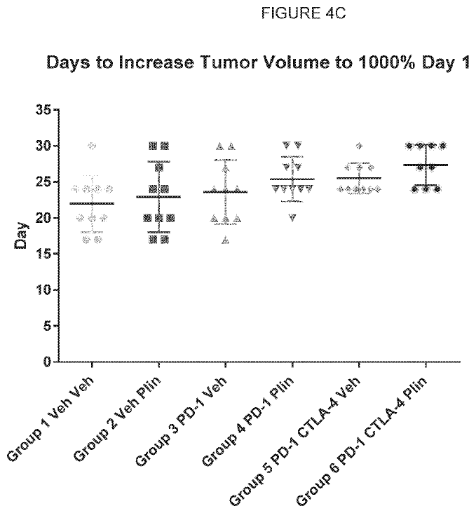

The test results of the combined treatment with Plinabulin and PD-1 antibody and CTLA-4-antibody showed that Plinabulin acted in synergy with the antibodies in inhibiting tumor growth and had the longest time to reach 10-fold increased tumor weight among the six test groups. FIG. 4A shows the effects of Groups 1, 5, and 6 on tumor growth. As shown in FIG. 4A, Group 6, the combined treatment with Plinabulin, PD-1 antibody and CTLA-4-antibody, had better inhibition of tumor growth than Group 5, the combination of PD-1 antibody and CTLA-4 antibody treatment group, and both groups 5 and 6 showed inhibition of tumor growth when compared with the control group 1. FIG. 4B shows the effects of the six treatment groups on the mean tumor weight at necropsy. As shown in FIG. 4B, the combined treatment with Plinabulin, PD-1 antibody and CTLA-4-antibody produced the lowest mean tumor weight at necropsy, followed by the treatment group with Plinabulin and PD-1 antibody. FIG. 4C shows the time for tumors to reach 10 fold of their starting volume in the six treatment groups. As shown in FIG. 4C, the treatment group with Plinabulin, PD-1 antibody and CTLA-4-antibody combined had the longest time for the tumors to reach 10 fold of their starting volume. Therefore, Plinabulin treatment either alone or in combination with PD-1 antibody or PD-1 plus CTLA-4 antibodies, resulted in a decreased tumor weight at necropsy. The combined treatment of Plinabulin, PD-1 antibody and CTLA-4-antibody had better tumor inhibitor effect than the treatment of Plinabulin and PD-1 antibody, which showed had better tumor inhibitor effect than the treatment of Plinabulin alone.

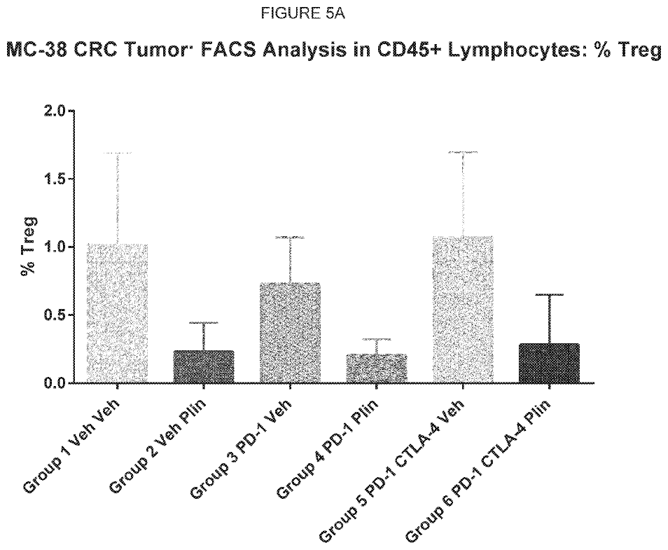

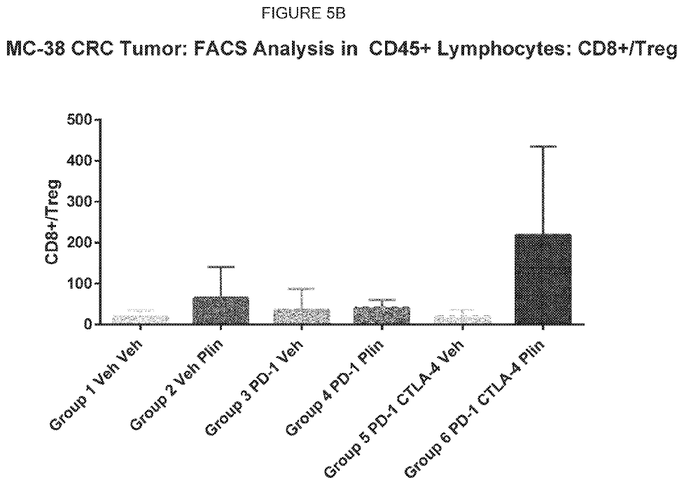

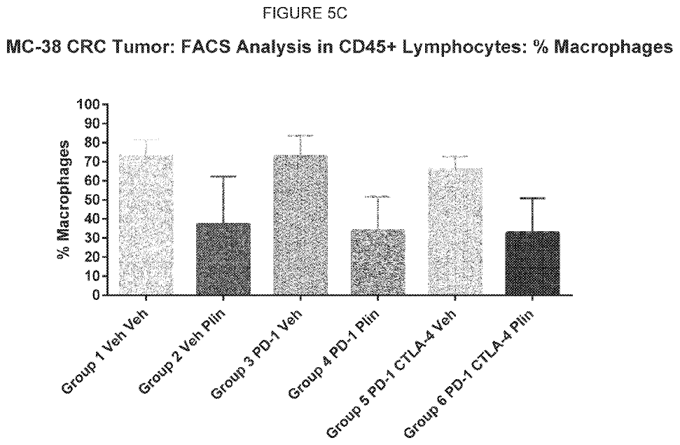

FIG. 5 shows the results of FACS analysis of the tumors at necropsy, including the percentage change of Treg cells, the ration of CD8+/Treg, and the percentage of macrophages in CD45+ lymphocytes, in the MC-38 CRC tumor model described above. FIG. 5A shows the effects of the six treatment groups on the percentage of Treg cells. As shown in FIG. 5A, the treatment of Plinabulin, PD-1 antibody and CTLA-4-antibody, the treatment of Plinabulin and PD-1 antibody and the treatment of Plinabulin alone all showed a reduction in % Treg cells as compared to the comparator group without plinabulin. FIG. 5B shows the ratio of CD8+ cells to Treg cells. As shown in FIG. 5B, the treatment of Plinabulin, PD-1 antibody and CTLA-4-antibody showed the highest ratio of CD8+/Treg cells. FIG. 5C shows the effects of the six treatment groups on macrophages. As shown in FIG. 5C, the treatment group of Plinabulin, PD-1 antibody and CTLA-4-antibody, the treatment group of Plinabulin, and the treatment group of PD-1 antibody and CTLA-4-antibody all showed decreased percentage of macrophage when compared with the respective comparator groups.

Therefore, the FACS analysis of the tumor tissue demonstrated that treatments of Plinabulin alone, Plinabulin and the immune checkpoint inhibitors (e.g., plinabulin with PD-1 antibody, Plinabulin with PD-1 antibody and CTLA-4-antibody) were associated with a decreased percentage of Regulatory T cells (Treg cells), a decreased percentage of macrophage stained cells, and a concomitant increase in the ratio of CD8+/Treg cells. The decrease of the Treg cells percentage and macrophage stained cells and the increase in the ratio of CD8+/Treg cells were more significant in the treatment groups with plinabulin and immune checkpoint inhibitors than the group with plinabulin alone or antibody(antibodies) alone. These data have demonstrated the synergistic immuno-oncology properties of the combined treatment using Plinabulin and the immune checkpoint inhibitors (e.g., PD-1 antibody and CTLA-4-antibody).

What is claimed is:

1. A method of therapeutically treating non-small cell lung cancer (NSCLC) in a human subject, the method comprising performing a therapeutic treatment cycle on the subject two or more times, each treatment cycle comprising:
administering a human monoclonal antibody that is a PD-1 inhibitor or a PD-L1 inhibitor to the subject on Day 1 of each treatment cycle;
administering plinabulin to the subject on Day 1 of each treatment cycle; and
administering docetaxel or a platinum-based agent to the subject on Day 1 of each treatment cycle.

2. The method of claim 1, wherein the treatment cycle is repeated more than two times.

3. The method of claim 1, wherein each treatment cycle comprises no additional administrations PD-1 inhibitor, PD-L1 inhibitor, plinabulin, docetaxel, or platinum-based agent.

4. The method of claim 1, wherein the human monoclonal antibody is selected from the group consisting of nivolumab, pembrolizumab, pidilizumab, BMS 936559, atezolizumab, and durvalimumab.

5. The method of claim 1, wherein the human monoclonal antibody is nivolumab.

6. The method of claim 1, wherein the human monoclonal antibody is pembrolizumab.

7. The method of claim 1, wherein the platinum-based agent is selected from the group consisting of oxaliplatin, carboplatin, and cisplatin.

8. The method of claim 1, wherein each treatment cycle is 21 days.

9. The method of claim 1, wherein each treatment cycle is 14 days.

10. A method of therapeutically treating non-small cell lung cancer (NSCLC) in a human subject, the method consisting of performing a therapeutic treatment cycle on the subject more than two times, each treatment cycle consisting of:
administering a human monoclonal antibody that is a PD-1 inhibitor or a PD-L1 inhibitor to the subject on Day 1 of each treatment cycle;
administering plinabulin to the subject on Day 1 of each treatment cycle; and
administering docetaxel to the subject on Day 1 of each treatment cycle.

11. A method of therapeutically treating non-small cell lung cancer (NSCLC) in a human subject, the method consisting of performing a therapeutic treatment cycle on the subject more than two times, each treatment cycle consisting of:
administering a human monoclonal antibody that is a PD-1 inhibitor or a PD-L1 inhibitor to the subject on Day 1 of each treatment cycle;
administering plinabulin to the subject on Day 1 of each treatment cycle; and
administering a platinum-based agent to the subject on Day 1 of each treatment cycle.

12. The method of claim 11, wherein the platinum-based agent is selected from the group consisting of oxaliplatin, carboplatin, and cisplatin.
